(19) United States
(12) Patent Application Publication
(10) Pub. No.: US 2019/0046055 A1
(43) Pub. Date: Feb. 14, 2019

(54) METHOD FOR OBTAINING USEFUL DATA ASSOCIATED WITH HEART RATE VARIABILITY PATTERN

(71) Applicant: Universidad De Sevilla, Sevilla (ES)

(72) Inventor: Alfonso Miguel GAÑÁN CALVO, Sevilla (ES)

(21) Appl. No.: 16/079,528

(22) PCT Filed: Feb. 23, 2017
(86) PCT No.: PCT/ES2017/070104

§ 371 Date: Aug. 23, 2018

(30) Foreign Application Priority Data
Feb. 24, 2016 (ES) P201600164

(51) Int. Cl. A61B 5/024; A61B 5/0205; A61B 5/0456; A61B 5/0464; A61B 5/044; A61B 5/04; G16H 40/63

(52) U.S. Cl. CPC A61B 5/02405; A61B 5/0205; A61B 5/0456; A61B 5/0816; A61B 5/044; A61B 5/04012; G16H 40/63; A61B 5/0464

(57) ABSTRACT

Method for providing a description, graphical representation, and a graphical identification of specific operating patterns of quasi-periodic cyclic systems, such as, but not limited to, reciprocating combustion engines, rotary machines, or biological organs such as the heart is disclosed. The disclosure also relates to a method for calculating an indicator evaluating the heart health or condition of an individual, as well as for diagnosing and issuing prognoses relating to the functionality, pathology, or standard of health of a machine or organism equipped with a motor or organ that operates cyclically, and to provide a description, a compact graphical representation, and a graphical identification of specific operating patterns of dynamic systems, for example economic systems such as the stock market.

### TECHNICAL FIELD

The invention that is proposed consists of a method of obtaining graphical data or the resulting multidimensional codes thereof, for characterizing and evaluating cyclic or quasi-periodical dissipative systems of any kind, whether natural or artificial, in a compact, universal, adaptable, and accessible manner. Said invention is essentially based on the following elements:
(i) A general formulation of the normalized and sequential variability of time intervals between subsequent cycles, using a key of various parameters, which determine the particular algorithmic expression of said sequential variability. This expression can also be considered a general transform of the original numerical series of the time intervals. Furthermore, this transformation is univocal and establishes a sequential partition of the original series, selecting subsequent groups of N elements in an order previously established by the chosen key. Said groups can be considered vectors in an N-dimensional space.
(ii) A spatial representation in N-dimensions of the vectors or points generated by the transformation defined by the selected key. In particular, for N=4, the three dimensions and color can be used. In the case N=5, it is possible to use the three spatial dimensions, color, and a partition of the fifth dimension which can be represented dynamically, for example by means of a time sequence (video) of the three-dimensional representation corresponding to each interval of said partition, using time as the dimension for representing the fifth dimension.
(iii) The identification of common spatial patterns or correlations (clusters) between groups of points, such as crystallographic planes or axes, general surfaces, etc., obtained through the representations of a sufficient number of systems of the same nature.
(iv) The comparison of an individual system of a certain nature with said common patterns, and the determination of the presence of said common patterns in the individual system to be evaluated.

Contrary to any other method using other transformations, such as the Fourier transform or the Wavelet transforms of any type, the proposed method is intrinsically adaptive and contemplates the accumulation of knowledge through experience. It furthermore allows using the instantaneous time scale or any other scale that may be locally selected, such as the main reference scale of the analysis; this allows identifying universal variability patterns, independent of other time scales or exogenous variables, which are inaccessible for other transformations such as the Fourier transform since they overlap all the time scales regardless of their sequence of appearance.

### BACKGROUND OF THE INVENTION

The cyclic dissipative systems are characterized by specific time periods, whether they are constant or variable. Therefore, they belong to a special class of dynamic systems the degrees of freedom of which are critically restricted or limited. Unlike what occurs with generic dissipative dynamic systems, which show chaotic behavior, cyclic dissipative system attractors are well defined and are generally simple. Such systems are generally mechanical or biomechanical motors which exchange energy inputs or demands or work with the environment, whether it is an automobile engine, a wind turbine, or a heart. However, whereas constant time periods characterize both, artificial clocks and natural motors, designed specifically to minimize variability, or not being subjected to changes in demands, motors subject to variable demands also have variable time periods. This strategy is the most cost-effective way to assure the adaptive capacity of an organism (for example, an animal) or a machine (for example, a car) equipped with these motors.

The adaptive capacity with respect to changes in demands is probably the main priority of natural and artificial systems equipped with movement capabilities. These systems are inherently dissipative. On the other hand, all the artificial systems designed to work under constant demand also need to accelerate from a pause or decelerate until stopping. The degree of adaptability, or of how a system responds to a given demand, which in turn has its own characteristic time, designates its force, robustness, or health, and generally determines its survival.

Adaptable mobile systems generally experience periods during which they are in standby: for example, the idling speeds of internal combustion engines to prevent frequent starts and stops, the sleep of animals, etc. During these periods of inactivity, the system extends the characteristic time period exclusively to balance out internal dissipation. However, the complexity of the most adaptable mobile systems requires their internal cyclic motors to have a limited number of degrees of freedom. These limited degrees of freedom, which assure adaptability, are almost incompatible with constant time periods. The variability shown by a system in inactive mode openly reflects its internal characteristics and describes its compromises, which are often concealed under a general operation. For example, the two-stroke engines show strong variability when they are idling, and heart rate variability can be observed during deep sleep or in a situation of deep relaxation much better than during grilling exercise in healthy subjects.

An ample series of methods have been proposed for characterizing dissipative dynamic systems: description of strange attractors, Lyapunov exponents, entropy analysis, power laws, Fourier analysis, multidimensional phase portraits, etc. However, none of these methods provides in-depth information or univocal portraits for cyclic and adaptive dissipative systems due to the inherent nature thereof. There is no compact equivalent to a QR code or a graph to provide complete information about the nature, adaptability, state of health and internal characteristics of a cyclic dissipative system.

A very general case of systems with high variability and adaptability is that of living organisms equipped with a circulatory system and mobility. The circulatory system of the living organisms is an autonomous mechanical system delicately coupled with the respiratory system, and both have developed through evolution in response to the complex patterns of oxygen demand associated with movement. Circulatory health is based on adaptive capacity, which entails inherent variability. In the embodiment of the present invention described below, an N-dimensional graph calculated by means of the described method and representing heart rate variability shows two universal arrhythmic patterns as specific signs of health: one reflects the adaptive capacity of the heart, and the other reflects the harmony between heart-respiratory rhythm. Furthermore, at least three universal arrhythmic patterns are identified, the presence of which increases progressively in a manner that is proportionally detrimental to the two patterns of health, in certain pathological situations (myocardial infarction, heart failure, and recovery after sudden death). The presence of the identified universal arrhythmic structures, together with the position of the center of mass of the heart rate variability graph, provide an unprecedented quantitative assessment of the pathology-health gradient.

The heart is the first organic autonomous volumetric pump developed by nature, and it allowed the enormous leap that mobility entailed for living organisms. The basic functions of the heart are contractility and heart rate. Heart rate variability (HRV) represents a vital degree of freedom of the evolution of an autonomous organism with a circulatory system (Malik, 1996) that allows the immediate adaptive response to oxygen demand. These demands can have innumerable variability profiles. However, due to principles of economy, nature responds to demands with the creation of a limited number of structures or patterns, instead of giving different responses to the innumerable possible solutions. Therefore, one must ask, among other questions, to what extent do predetermined structures or patterns appear in HRV? Might some of those structures reveal general modes of adaptability (health) or of failure (pathology)? Can they appear combined? What would their general relative size and weight be with respect to the rest of the events? Are they complementary or antagonistic structures?

The respiratory system, which is complementary to the circulatory system in mobile organisms that live in the air, is also a volumetric pump (formed by the chest and lungs) with another characteristic time and an operation with a significant level of coupling to the cardiac system. The flexible relationship between heart rate and respiratory rate entails a specific HRV. The heart rate is also subjected to other endogenous influences, such as digestion, age and sex, biochemical mechanisms, or psychic activity, the characteristic times of which are decoupled from the autonomous control of the heart rate. When such influences reach or exceed external demands (including the circadian cycle), the organism may present pathological arrhythmias. However, those that are the most life-threatening are the ones that have rates greater than the respiratory rate. An example of the application of the present invention described herein shows that in the human species, there can be universal specific arrhythmic sequences (internal structure) as a co-evolutionary product of the autonomous (sympathetic-parasympathetic) nervous system, and that those sequences can be specific to a healthy heart or a diseased heart.

The need for precise and specific non-invasive tools for diagnosis and prognosis is a critical factor for advancement in medicine. Compact graphical representations of physiological systems have been of enormous help for doctors as regards the details and precision in diagnosis. Imaging internal systems and tissues (for example, ultrasound, computerized axial tomography, or magnetic resonance imaging (MRI)) has changed lives. As a result, in cardiac quantification (CQ), echocardiography (echoCG) has represented an extremely important development. Nevertheless, the series of electrocardiographic data provide irreplaceable complementary information that is inaccessible for echoCG in many aspects, such as the occurrence of patterns and temporary pathological behaviors, ectopic rhythms, etc. The reduction of the time series of a heart rate Holter recorder (HR) to a compact graphical representation (or graph) can have a clinical value that is complementary to that of the echoCG. Preliminary considerations are necessary: for the purpose of determining the occurrence of generic patterns reflecting normal or pathological characteristics and for improving their specificity, the graph must reduce to a minimum (i) the individual differences in weight/size, sex, age, etc., (ii) the long-term influences on heart rate, with times comparable to or greater than those of the respiratory rate, and (iii) both exogenous and endogenous influences, which are different from those of a cardiac or circulatory origin. The graphical representation that is sought must provide complete information provided by the sequence of all cardiac events (for example, the complete sequence beat by beat, or the RR intervals). After a thorough review, there is no evidence of there having been any representation with all those characteristics sought up until now.

Among the different approaches for studying HRV, including tools for the analysis of dynamic systems and chaos, multiscale entropy analysis, frequency domain analysis, and Markov chains, Poincaré maps of RR intervals provide compact graphs useful for the HRV analysis. The high stochastic heterogeneity HRV generally has is an indication of the existence of strong, presumably universal, internal structures. Under this premise, the most suitable tool for HRV would be a normalized time sequence analysis, as opposed to global stochastic tools or Fourier transforms. In fact, Fourier transform can definitively be excluded as a tool suitable for analyzing the universal variability patterns, since it essentially analyzes the content of fixed time (or frequency) scales in the entire recording, such that it mixes patterns of the same potential nature for high and low heart rates in the same HR.

The Poincaré representation (or recurrence maps) provides a direct sequence analysis of the RR series for identifying time patterns. However, the limitations of the representation of Poincaré recurrence maps in the two-dimensional (2D) spatial plane conceal a large part of the potential of graphs of this type.

Another type of representation is described, for example, in patent document U.S. Pat. No. 4,934,374 (Ostlund et al.), where the represented object is the proximity between vectors defined by successive values of a signal with a given time delay as a function of time. The method described in this document is based on representing the time variable on both axes of a two-dimensional map, but the spatial representation of which continues to be planar (one-dimensional). Likewise, the representation described in said patent is based on a concept of “variability masked” by the modulus of a non-universal vector. Therefore, the influence of the instantaneous physical state of the patient will rule out any reliable form of detection of universal patterns.

Although this representation allows distinguishing ordered cyclic, quasi-periodical, or chaotic systems, it does not allow distinguishing complex sequences over time that are repeated or are specifically associated with certain pathologies. These characteristics are associated, for example, with specific arrhythmic sequences which appear, in any planar representation (as in the case of U.S. Pat. No. 493,374), overlapping and indistinguishable, the identification thereof in evaluation or diagnostic phases thereby being prevented.

Another example of a planar representation is the registration for the patent application published as US 2005/0171447 A1 (Esperer), which claims the use of the conventional two-dimensional Poincaré recurrence map. The author of that invention uses information in addition to that provided by the planar representation (the invention particularly uses the local density of points in each area of the plane that is represented) to correlate results with known pathologies. Nevertheless, this representation unavoidably conceals, by being superimposed on the plane, many complex multidimensional sequences which, again, the present invention does in fact show as a specific characteristic.

An example of multidimensional representation is shown in patent application PCT/GB2015/050429, where the represented object is a multidimensional vector consisting of successive values of any one signal coming from a quasi-periodical or cyclic system, separated by a fixed amount of time τ between every two successive values. The result is a more or less complex multidimensional orbit, the interpretation of which in terms of the identification of given complex multidimensional sequences may be impossible due to the inevitable possibility of there being multi-evaluated areas for one and the same sequence, which would prevent reconstructing said sequence. Even by varying the value of τ and producing a collection of different representations, the identification of complex sequences can be so difficult that a huge computational effort would be required to compact the information that is sought. Furthermore, said information must subsequently be correlated with the information about associated pathologies. In comparison, the selection of the values of the multidimensional vectors proposed in the present invention is the most suitable for offering the most compact and direct representation of complex sequences, which can be viewed directly without the need for any subsequent computational effort. Already known shape identification algorithms (which are not the object of the present invention) can then be applied to produce useful correlations between shape and pathology in a reliable, reproducible, compact, and quick manner.

In summary, in the present technical field it is necessary to develop new methods for the compact representation of the pattern of heart rate variability, which allow obtaining more detailed information than that which is generated by the methods used up until now. The present invention seeks to solve the aforementioned need by means of a novel method for the univocal and compact representation of cyclic or quasi-periodical dissipative systems of any nature which are of particular use in the analysis of cardiac patterns. Likewise, said method does not depend on the time of obtaining data, and by means of its steps of normalization and compaction, it allows finding associated patterns and the evolution thereof regardless of the time scale and in a multidimensional manner, which is not possible in other representations, such as those described in U.S. Pat. No. 4,934,374, US 2005/0171447 or PCT/GB2015/050429.

### BRIEF DESCRIPTION OF THE INVENTION

A first object of the present invention relates to a method for providing a description, compact graphical representation, and a graphical identification of specific operating patterns of quasi-periodic cyclic systems, such as, but not limited to, reciprocating combustion engines, rotary machines, or biological organs such as the heart, characterized by:

a) measuring and recording a number M of consecutive time intervals {Xᵢ}_{i=1, . . . ,M} corresponding to cycles of one or more components of a heart “pQRSt” complex of an electrocardiogram, with a precision equal to or greater than 10% of the mean value of the cycle time, and M being greater than 2;

b) calculating the variability on said M intervals of a sequence of consecutive vectors {δⱼ}_{j=1, . . . ,M-N} of N components, according to the algorithm or transformation defined by the expression:

$${δ}_{j} = {\{ {\sum }_{n = 0}^{m} ( \begin{matrix}m \\ n\end{matrix} ) {( - 1 )}^{n} ( \begin{matrix}{〈 X 〉}_{{N}_{0} , j + {K}_{0}}^{- 1} {X}_{j + n + k \cdot {ɛ}_{0} + {J}_{0}} - \\ {ɛ}_{1} {〈 X 〉}_{{N}_{1} , j + {K}_{1}}^{- 1} {〈 X 〉}_{{N}_{2} , j + n + k \cdot {ɛ}_{2} + {K}_{2}}\end{matrix} ) \}}_{k = {J}_{1} , … , {J}_{1} + N - 1} ,$$

with the following notation:

$${〈 X 〉}_{L , l} = {L}^{- 1} {\sum }_{h = 0}^{L - 1} {X}_{l + h} , with ( \begin{matrix}m \\ n\end{matrix} ) = \frac{m !}{n ! ( m - n ) !} .$$

The following parameters are integers and their selection determines the final form of the mentioned transformation:

{m,N,N₀,N₁,N₂,ε₀,ε₁,ε₂,ζ₀,ζ₁,J₀,J₁,K₀,K₁,K₂},

where:
m is a natural indicator representing the order of the discrete variation that is calculated;
N is the dimension or number of components of each vector δⱼ, where N≥2;
N₀,N₁, and N₂ indicate the number of values that are used for calculating the corresponding indicated local average in the general formula of the algorithm;
ε₀, ε₁, ε₂ have binary values 0 or 1, and indicate if the corresponding elements are, respectively, fixed or mobile in the calculation of each of the components of the vector δⱼ;
ζ₀, ζ₁ have binary values 0 or 1, and indicate if the local mean is, respectively, fixed or mobile;
J₀ and J₁ indicate the delay or advance of the first element that is taken in the calculation on the basis of the indicator j;
K₀, K₁, K₂ indicate the delay or advance of the first element that is taken in the corresponding local series for calculating the indicated local average.

The second object of this invention is the graphical representation in two or more dimensions of the position of the point indicated by the values of the components of each of the vectors δⱼ calculated as described above, for example, but without limitation, using the two spatial dimensions of a display, the numerical value of the color, and the size of a circular marker for representing four dimensions, or five dimensions if axonometric or conical projections in the plane are used, which in this case allows the user to view the volumetric extension of the three-dimensional representation generated for the identification of specific graphical patterns shown by the studied system.

A third object of the present invention is the automatic determination of the existence of behavioral patterns defined by a vector function A={aⱼ}_{j=1, . . . ,N}, where the elements aⱼ are specific fixed values or functions of one or more variables defined by the user, without any limitation, according to the following method:
a. Calculating the general angle θᵢ the cosine of which is determined by:

$$cos ( {θ}_{i} ) = \frac{A \cdot {δ}_{i}}{‖ A ‖ \cdot ‖ {δ}_{i} ‖} ,$$

where the vectors δⱼ are calculated as described above, and the symbol means the general norm of a vector in N dimensions, such that:

$$‖ A ‖ = {( {\sum }_{j = 1}^{N} {a}_{j}^{2} )}^{1 / 2} .$$

b. Calculating the number of events M′ such that the angle θᵢ is less than a predetermined tolerance ε, where 0<ε<1, preferably 0<ε<0.1, such that the function A must be explored in its space of existence in order to find said events in which θᵢ<ε; i.e., M′ depends on the selection of the specific values of the variables of the function A. In particular, if A is constant, M′ is unique.
c. Using the coefficient M′/M as an indicator evaluating the system according to if it shows predetermined behaviors defined by the vector A to a greater or lesser intensity, where said behaviors can be wanted or unwanted.

Another object of the present invention is also a method for the quantitative determination of the adaptability of machines or quasi-periodic cyclic systems, characterized by:
a. Subjecting the machine or system to a variation in operating conditions, due to a progressive increase or decrease in energy consumption, measuring the consecutive values Xᵢ of the time intervals corresponding to each cycle.
b. Using the coefficient M′/M, calculated according to the mentioned third object of this invention, on the basis of the series {Xᵢ}_{i=1, . . . ,M} with the specific definition of the vector A=t{(N+1)/2−j}_{j=1, . . . ,N}, where N>1, and said vector would correspond to a homogenous acceleration or deceleration of the system if t=1 or t=−1, respectively.

Another object of this invention is a method for calculating an indicator evaluating the heart health or condition of an individual, characterized by:
a. Measuring and recording a number M of consecutive time intervals {Xᵢ}_{i=1, . . . ,M} between peaks “R” of the heart “pQSRt” complex with a precision that is better than 10 ms, M being greater than two;
b. Calculating the series {δⱼ}_{j=1, . . . ,M-N} of consecutive vectors of N dimensions or components as described in the first object of this invention, calculated according to the following definitions of the fifteen parameters:

$$\{ \begin{matrix}m = 0 \\ N = 5 \\ {N}_{0} = 5 \\ {N}_{1} = 5 \\ {N}_{2} = 5 \\ {ɛ}_{0} = 1 \\ {ɛ}_{1} = 1 \\ {ɛ}_{2} = 0 \\ {ς}_{0} = 1 \\ {ς}_{1} = 1 \\ {J}_{0} = 0 \\ {J}_{1} = 0 \\ {K}_{0} = 0 \\ {K}_{1} = 0 \\ {K}_{2} = 0\end{matrix} \} ,$$

such that the definition of the calculation algorithm according to the first object of this invention is finally δⱼ={Xⱼ₊ₖ [symbol] X [symbol] _{N,j}⁻¹−1}_{k=0, . . . ,N-1}.

In the same sense, another object of this invention is a method for calculating an indicator evaluating the heart health or condition of an individual, characterized by:
a. Calculating the number of events M′ according to the third object of this invention on the basis of the series {Xᵢ}_{i=1, . . . ,M} with the specific definition of the vector A=t{(N+1)/2−j}_{j=1, . . . ,N}, where N>1.
b. Using the indicator m_{S1}/M, with m_{S1}=M′ calculated in the preceding point, directly or in combination with any other indicator for evaluation, for determining the level of heart health, for example but not limited to the use of m_{S1}/M as a direct indicator for evaluating heart health.

Likewise, another object of this invention is a method for calculating an indicator evaluating the heart health or condition of an individual, characterized by:
a. Calculating the number of events M′ according to the third object of this invention on the basis of the series {xᵢ}_{i=1, . . . ,M} with the specific definition of the vector A_{N}=t{sin(2π·j)/N}_{j=1, . . . ,N}, where N can range from N=3 to N=12, which corresponds to a sinusoidal modulation of the heart rhythm combined with the respiratory rhythm, where t can have any value, for example, but without limitation, 1 or −1.
b. Using the indicator m_{S2}/M, with m_{S2}=M′ calculated in the preceding point, directly or in combination with any other indicator for evaluation, for determining the level of heart health, for example but not limited to the use of m_{S2}/M as a direct indicator for evaluating heart health.

Likewise, another object of this invention is a method for calculating an indicator evaluating the heart health or condition of an individual, characterized by:
a. Calculating the coefficient M′/M according to the third object of this invention on the basis of the series {xᵢ}_{i=1, . . . ,M} with the specific definition of the vector

$${A}_{N} = t \{ - 1 , 1 , \underbrace{0 , … , 0}_{N} \} ,$$

where N can range from N=1 to N=20, corresponding to a compensated ectopic beat, and where t can have any value, for example, but without limitation, 1 or −1.
b. Using the indicator m_{E}/M, with m_{E}=M′ calculated in the preceding point, directly or in combination with any other indicator for evaluation, for determining the level of heart health, for example but not limited to the use of m_{E}/M as a direct indicator for evaluating heart health.

Likewise, another object of this invention is a method for calculating an indicator evaluating the heart health or condition of an individual, characterized by:
a. Calculating the coefficient M′/M according to the third object of this invention on the basis of the series {Xᵢ}_{i=1, . . . ,M} with the specific definition of the vector

$${A}_{N} = t \{ N , \underbrace{- 1 , … , - 1}_{N} \} ,$$

where N can range from N=2 to N=20, corresponding to a regular paroxysmal tachycardia, and where t can have any value, for example, but without limitation, 1 or −1.
b. Using the indicator m_{TP}/M, with m_{TP}=M′ calculated in the preceding point, directly or in combination with any other indicator for evaluation, for determining the level of heart health, for example but not limited to the use of m_{TP}/M as a direct indicator for evaluating heart health.

Another object of the present invention is any use for diagnosing the functionality, pathology, or level of quality or health of a machine or organism equipped with a motor or organ that operates cyclically, using any of the methods described above.

Another object of this invention is any use used for issuing prognoses about the future functionality, pathology, or level of quality or health of a machine or organism equipped with a motor or organ that operates cyclically, using any of the methods described above.

A final object of this invention is any use for the description, compact graphical representation, and graphical identification of specific operating patterns of dynamic systems, such as, but not limited to, economic systems such as the stock market, using any of the methods described above applied to:
a. Sequential series of values {Xᵢ}_{i=1, . . . ,M}, obtained by measuring the value of a certain characteristic amount of the system at regular time intervals or with a given frequency.
b. Sequential series of values {Xᵢ}_{i=1, . . . ,M}, obtained by measuring the value of a certain characteristic amount of the system following given time guidelines, that are not necessarily regular or constant, for the acquisition or measurement of the values Xᵢ.

### DESCRIPTION OF THE DRAWINGS

FIG. 1. Representation of the distance of the vectors for a particular case of heart failure. Characteristics lines of this specific case and their mathematical expressions are shown. Furthermore, central regions of the graph have been enlarged with the areas of interest highlighted in order to see the details.

FIG. 2. (a) Four-dimensional graph of the location of M−3 vectors {Δᵢ}_{i=1, . . . ,M-N+1} normalized with the overall mean of a normal HR (a healthy adult). (b, c) The same for two subjects with chronic heart failure (three viewing angles of each). The graphs located on the right in (b, c) show universal patterns when projected with the suitable angle. (d) Identification of the main lines of the heart failure in two different individuals with HF.

FIG. 3. (a) The different regions of normal activity and of HF found in this study: NSR (normal activity, green; Fantasia, cyan); HF (magenta). (b1-b3) The different regions occupied by the four situations found in this study in the multivariable space {log₁₀(A1),log₁₀(B1),log₁₀(Φ_{N})}: NSR (n.a., green; Fantasia, cyan); MI (blue); SD (red); HF (magenta). For greater clarity, the left panel provides three situations (NSR n.a., myocardial infarction and HF), the central panel provides four situations (NSR n.a., MI, SD and HF), and the right panel provides all the situations studied. N=5 in all the results of this Figure.

### DETAILED DESCRIPTION OF THE INVENTION

A map of Poincare is a graph consisting of the representation of a recurrence map or sequential path of the values having a certain variable in consecutive cycles. Specifically, a two-dimensional (2D) recurrence map is a planar projection where complex paths, with multidimensional characteristics (i.e., specific arrhythmic sequences) are overlapping and indistinguishable. This invention allows for a generalization of said recurrence maps. Among other possibilities, a normalized variability using a moving average of order N=5 can be formulated. This formulation corresponds with the following selection of the fifteen values defining the base algorithm of the methods described in this invention:

$$\{ \begin{matrix}m = 0 \\ N = 5 \\ {N}_{0} = 5 \\ {N}_{1} = 5 \\ {N}_{2} = 5 \\ {ɛ}_{0} = 1 \\ {ɛ}_{1} = 1 \\ {ɛ}_{2} = 0 \\ {ς}_{0} = 1 \\ {ς}_{1} = 1 \\ {J}_{0} = 0 \\ {J}_{1} = 0 \\ {K}_{0} = 0 \\ {K}_{1} = 0 \\ {K}_{2} = 0\end{matrix} \} .$$

By means of this selection, referred to as NL (Local Normalization), an expression of the sequential variability {δⱼ}_{j=1, . . . ,M-4}, with

$${δ}_{j} = {\{ {X}_{j + k} {〈 X 〉}_{N , j}^{- 1} - 1 \}}_{k = 0 , … , 4} , {〈 X 〉}_{N , j} = {\sum }_{i = 0}^{N - 1} {X}_{j + i} ,$$

is obtained and the representation thereof meets the aforementioned requirements for an ideal representation of the heart function in terms of compactness and integrity, and furthermore it reduces to a minimum exogenous and accidental influences as a result of the local normalization resulting from the values {Xᵢ}_{i=1, . . . ,M}. The proposed method can be applied to any of the components of the heart “pQRSt” complex, although this example focuses on the analysis of the main component, the series of RR intervals. Generally, by exploring the interval of order from N=2 to N=100, it can be seen that a quantification measurement of the overall variability, such as the norm or argument of the center of mass vector of the distribution of points defined by the vectors δⱼ={Xⱼ₊ₖ [symbol] X [symbol] _{N,j}⁻¹−1}_{k=0, . . . ,N-1}≡{_{j,k}}, recurrently has a minimum for N=5 in the case of healthy individuals. This could be due to the fact that the average heart rate is about five times greater than the respiratory rate in the human species, which gives rise to a subharmonic of order five of the HRV, thereby maximizing overall compensation when N=5. If this hypothesis is correct, deviations from normality should optimally be distinguished using the order N=5.

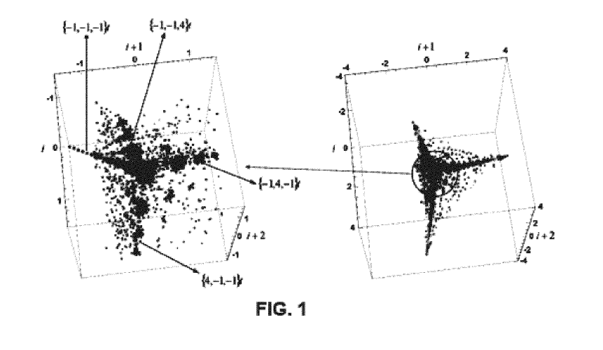

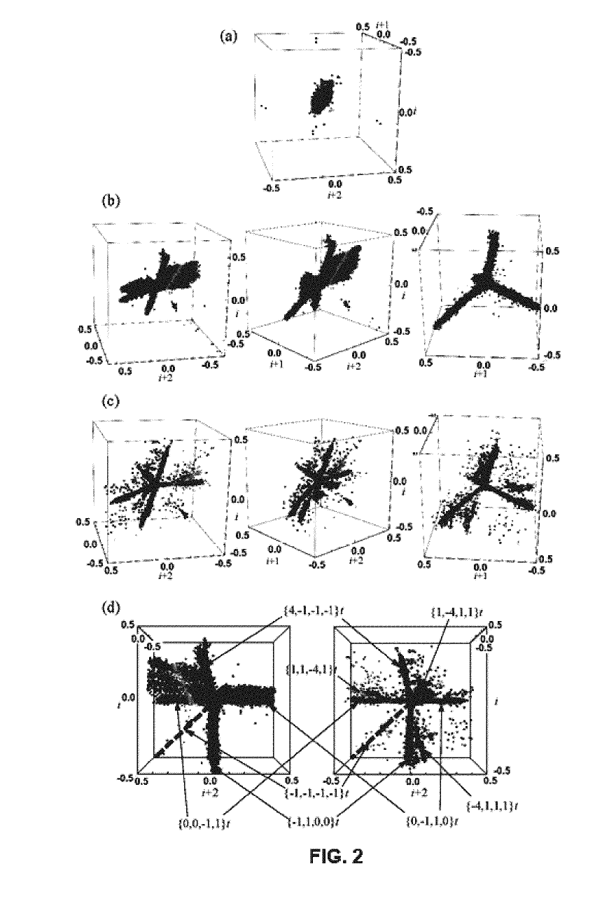

Fortunately, N=5 gives rise to the most complete graph, with complete graphical representation possibilities, among all the possible orders. In fact, the expression δⱼ={Xⱼ₊ₖ [symbol] X [symbol] _{N,j}⁻¹−1}_{k=0, . . . ,N-1} allows the representation thereof in N−1 dimensions since the information contained in N−1 of the total of N elements provides all the information of the vector of N dimensions. That is because the value of any of the N elements can be obtained on the basis of the other N−1 since the sum of the N elements is always nil, by definition. The resulting four-dimensional sequential information vector, which can be graphically represented in 3D plus color, gives rise to a valuable descriptive and comparative tool from the clinical point of view. This graph allows the identification of sequences that originate universal patterns, the distribution and density of which provide an immediate and complete information about the state of cardiac function. Up to five universal sequences, which can particularly be seen in the graphical representation (see an example in FIG. 1, with N=4, and other examples in FIG. 2, with N=5 and another selection of parameters), have been identified using the method described, but they are not only present when N=5: they can all be found in higher orders, and their general expressions for any order N have in fact been obtained.

The universality of these sequences has been verified in a series of HR databases, with 133 records clustered into in four basic situations with distinctive characteristics: (i) individuals in normal sinus rhythm (NSR) during normal activity, or at rest in the supine position while watching the film “Fantasia”; (ii) ischemic cardiopathy, specifically myocardial infarction (MI); (iii) heart failure (HF); and (iv) recovery from sudden death (SD). By applying the mentioned databases to it, the percentage of occurrence of each of the five sequences and the primary variability (the definition of which will be provided below) provide a univocal and specific set of measurements capable of assessing the state of the heart in the records included in the publically available databases that were used (see databases used below).

A Holter recorder (HR) is a set M of consecutive values corresponding to the RR intervals, which can be expressed as {Xⱼ}_{j=1, . . . ,M}. It is easy to demonstrate that the general distance vector in N dimensions from any point {Xⱼ₊ₖ}_{k=0, . . . ,N-1} formed by N RR intervals to the line of identity (line of zero variability, Piskorski & Guzik 2012), defined by the identity vector {1, 1, . . . , 1}_{N}, is defined as

$${D}_{j} = {\{ {X}_{j + k} - {〈 X 〉}_{N , j} \}}_{k = 0 , … , N - 1} .$$

The sequence of RR intervals of a healthy heart “would dance” around the line of identity but would never be supported on it (even in the extreme situations of complete relaxation or extreme exercise, there is a variability mathematically different from zero, even though the RRs are apparently constant). The locally normalized expression of said general distance vector is precisely the expression of the selection of NL parameters, i.e., δⱼ≡ [symbol] X [symbol] _{N,j}⁻¹Dⱼ={ [symbol] X [symbol] _{N,j}⁻¹ Xⱼ₊ₖ−1}_{k=0, . . . ,N-1}, where the basis for normalization is the local mean

$${〈 X 〉}_{N , j} = {\sum }_{i = 0}^{N - 1} {X}_{j + i} .$$

In FIG. 1, a simple color code ranging between 0 and 1 (the Hue code of the Mathematica® 9.0 program) has been used as a fourth dimension.

Another selection of parameters that has been used for this study is the following:

$$\{ \begin{matrix}m = 0 \\ N = 5 \\ {N}_{0} = 5 \\ {N}_{1} = M \\ {N}_{2} = M \\ {ɛ}_{0} = 1 \\ {ɛ}_{1} = 1 \\ {ɛ}_{2} = 0 \\ {ς}_{0} = 0 \\ {ς}_{1} = 0 \\ {J}_{0} = 0 \\ {J}_{1} = 0 \\ {K}_{0} = 0 \\ {K}_{1} = 0 \\ {K}_{2} = 0\end{matrix} \} ,$$

which gives rise to the following expression δⱼ={ [symbol] X [symbol] _{M}⁻¹Xⱼ₊ₖ− [symbol] X [symbol] _{M}⁻¹ [symbol] X [symbol] _{N,j}}_{k=0, . . . ,N-1}. This selection of parameters shall be referred to as NG (Overall Normalization), and the particular expression of δⱼ for this selection will be referred to as Δⱼ. This expression has the following trivial relations with Dⱼ={Xⱼ₊ₖ− [symbol] X [symbol] _{N,j}}_{k=0, . . . ,N-1} and with

δⱼ={Xⱼ₊ₖ [symbol] X [symbol] _{N,j}⁻¹−1}_{k=0, . . . ,N-1}.

Δⱼ= [symbol] X [symbol] _{M}⁻¹Dⱼ, and Δⱼ= [symbol] X [symbol] _{M}⁻¹ [symbol] X [symbol] _{N,j}δⱼ.

FIG. 2 shows the graphical representation of three HRs, analyzed with the proposed method, for N=5: FIG. 2 (a) represents a healthy subject, and FIG. 2 (b, c) represents two patients with chronic heart failure. While in the healthy individual it gives rise to a dense and compact shape around the origin, individuals with HF show distinctive spatial lines that follow previously established sequences. In view of this figure, some immediate conclusions can be extracted. First, the lines shown are fundamentally straight. In some cases (for example, FIG. 2 (b)), the space between two lines is joined by a characteristic plane, but there is a special viewing angle (angle of projection over the viewing plane) which systematically reduces the main straight lines and planes to just three in all cases [see FIG. 2 (c)].

As would be expected, the normalized Poincare sections of the variability of the NSR database (apparently non-pathological) are relatively centered and homogenously distributed around zero, showing a mathematical compensation (apparently) with certain randomization, i.e., a more or less spherical nucleus with a random, approximately Gaussian distribution. A more detailed observation shows that all the cases show a clear structure in the fourth dimension (color), along a specific direction. It is important to point out that this direction is exactly the same for all subjects, regardless the shape of the distribution of points representing the RR intervals. This fact points towards the existence of a subharmonic, probably related to the respiratory cycle, that reflects an automatism of the sympathetic-parasympathetic system. This effect is relatively rare or virtually absent in individuals with IM and is not found in individuals with HF and SD. The existence of an individual in the NSR database (around 12%) having the characteristics present in HF is quantified and discussed below, and it supports the fact that the presence of the sinus rhythm does not rule out the presence of a heart pathology.

The “Fantasia” database shows the same characteristics (lyengar et al. 1996; Schimitt et al. 2007) as the individuals in the NSR database, and almost the same percentage of subjects with HF characteristics. Furthermore, elderly healthy subjects clearly show less variability than young healthy subjects.

Almost 70% of the subjects in the HF database (Baim et al. 1986), and many of the subjects studied in the SD database (Taddei et al. 1992) show the same distinctive lines mentioned above. The percentage of subjects with homogenously distributed point clouds is proportionally inverse when compared with the individuals in the NSR database: less than 20%. On the other hand, the repetition of the patterns and their extent comply with the universal characteristics of HRV. Finally, individuals in the SD database used in this study show variable patterns that are completely different and visibly more irregular than any other group. Some of them have the same lines as the individuals with HF, but most of them show a very complex and apparently chaotic structure.

A first characteristic of the graphical representation proposed in this invention with both the NL and the NG selection (δⱼ or Δⱼ) is that it is centered around the origin by definition, such that the density of points in different areas can be considered a specific identifying signature of variability. Therefore, the norm of the normalized vector defining the center of mass of the graphical representation would be a primary measurement of the variability for an n-th subharmonic order, and of its overall degree of compensation (for example, in an HR of 24 hours for a circadian cycle). However, experience has shown that the entire record of vectors Δⱼ gives rise to an inadequate compensation due to the inherent nature of the Poincare representation: it can be seen that the same arrhythmic RR interval Xⱼ appears as a common component in N−1 vectors Δⱼ₋ₖ around the line of identity, which often produces an apparent overall compensation. To avoid this, a subset of all the series can be extracted by taking the indicator j in hops of N elements (i.e., j={1, 1+N, 1+2N, . . . }), where each Xⱼ appears only once. The distance from the center of mass of this subgroup to the origin, the graphical representation of which is virtually indistinguishable from that of the entire series except for the different densities thereof, is determined by:

$${Φ}_{N} = {[ {\sum }_{k = 1}^{N - 1} {( {\sum }_{i ; N}^{M - N + 1} {Δ}_{i , k} )}^{2} ]}^{1 / 2} ,$$

where Δ_{i,k} is the component k of the vector Δᵢ, and the notation of the sum

$${\sum }_{i ; N}^{M - N + 1} {Δ}_{i , k}$$

indicates N by N hops in the indicator i. This coefficient, referred to as primary variability (PV), can be represented for each individual as a function of N. Experience shows that Φ_{N} is about 10 to 100 times greater than the distance to the origin of the center of mass of the complete original set, which greatly amplifies the significance of Φ_{N} as it has been defined. It also depends on the number of beats in the series. In general, the minimum variability corresponds to NRS (Fantasia) individuals, closely followed by the variabilities of individuals with MI. The maximum variability corresponds to SD and HF, the distribution thereof being fairly similar. The variability of subjects in the NSR database with normal activity is at intermediate values. Φ_{N} is shown as a new quantitative measurement with extraordinary capacity to differentiate among patients with different heart function disturbances, in combination with the proposed graphical representation and arrhythmic structures derived from it.

A first classification of the different patterns that can be clustered into universal lines or sequences is provided below. On the other hand, it will be demonstrated that the density of points along those lines provides a valuable measurement of the state of the heart function. In fact, when the method of the invention is applied to the databases used, which are freely available, it gives rise to results with a high descriptive specificity for each group. As a first approach, the present invention is limited to the identification of the simplest primary arrhythmic sequences, which can be expressed in the form of a line, defined by the vector

A_{N}=t{aₚa₂, . . . ,a_{N}},

parameterized by an arbitrary variable t. Logically, the position vector of a real point (beat) on a specific arrhythmic line may correspond to any positive real value t. This reflects the intrinsic capacity of the described method: an arrhythmic line (or anomaly), which can be mathematically and universally expressed, brings together all the arrhythmias of the same nature, regardless of the heart rate and amplitude of variability. Several primary sequences that will be described below have been deciphered, and the density of points along the corresponding lines (sequences) has been calculated. These results do not exclude the existence of other more complex sequences, with specific associated characteristics that will be determined in future studies.

An immediate way to evaluate the density of points corresponding to a specific sequence is to quantify their presence in % through the entire total record of the series of RR. Given that the presence of a specific sequence is obviously not an exact or uniform amount in all the situations or in all individuals, it is necessary to use statistical means to determine the higher or lower presence of said sequence in a given heart condition. A convenient way to represent the distribution of the presence of a sequence in a certain condition is to determine the value of Fᵢ=i/M_{B} versus yᵢ for the corresponding database, yᵢ being the percentage of presence of the particular sequence A_{N}=t{a₁, a₂, . . . , a_{N}} of an individual, i being the range of said individual in particular, based on his or her score yᵢ, and M_{B} being the total number of individuals in the database. This would be determined for each sequence and analyzed in combination for each situation.

Arrhythmia A1:

a healthy individual with NSR must show an intrinsic capacity for responding to any demand of the organism, by means of regular accelerations and decelerations of the heart rate directed by the sympathetic/parasympathetic balance. This capacity must be reflected in the appearance of the simplest form of HRV, which can be expressed as a linear ramp:

A1=t{(N+1)/2−j}_{j=1, . . . ,N}

where the positive or negative sign of t is applied to the acceleration or deceleration of the heart rate, respectively. For example, for N=4, with an accelerated heart rate, it would be formulated as A1₄⁺=t{1.5,0.5,−0.5,1.5}, with t>0; for N=5 and a decelerated heart rate, it would be A1₅⁻=t{−2,−1,0,1,2}, with t>0, etc., where the higher values of t indicate a more abrupt rise or fall, or a more pronounced ramp, of the heart rate, without changes in the functional structure of the variability.

This is the dominant form of heart rate variability (HRV) in normal individuals in the MIT-BIH NSR database, as shown in Table 1. In fact, this is reflected in a slightly ellipsoid shape around the origin in the direction of the line of A1₅ of any four-dimensional graph of a Poincaré map of the fifth order, which represents the HRV of a normal individual with NSR, as has been proposed herein [see FIG. 2 (a)].

Even more importantly, as the presence of this form of HRV decreases, other forms of pathological arrhythmias, which will be described below, increase by about the same proportion. This finding fundamentally points towards a basic organic fact, i.e., this arrhythmia is actually the basic degree of freedom of the HRV and reflects the adaptive capacity of a healthy organism. If a pathological situation depresses or limits this degree of freedom, other forms of HRV will manifest to compensate for this deficiency. It is important to point out that these alternative forms are not arbitrary, particularly in the individuals included in the HF database. In fact, they can be considered universal. Accordingly, the specific form of these alternative HRV patterns must be linked with the specific condition of an organism, which opens up the door to new forms of rapid, non-invasive diagnosis.

Arrhythmia B1 (Compensated Ectopic Beat):

The four-dimensional graph of HR belonging to individuals with HF generally have three lines (FIG. 2 (d)) which can be easily identified as (see FIG. 2 (d)):

B1_{5,1}=t{−1,1,0,0},B1_{5,2}=t{0,−1,1,0},B1_{5,3}=t{0,0,−1,1}

First of all, these three sequences actually pertain to a single class of the following type

t{ . . . ,0,0,−1,1,0,0, . . . }

Second of all, the mean value of the four components is zero for any N>2, and these sequences can therefore be considered “compensated.” This means that the final point of the Poincare section of the n-th order, rests approximately on the line of identity. In other words, the arrhythmic sequence can also be considered “closed” or “concluded” since the last interval lasts for the same time as the local mean. The fact that the number of intervals with zero variability surrounding the succession {−1, 1} in most cases is greater than two (i.e., the line {1, 0, 0, −1} does not clearly appear) should be pointed out. It can therefore be concluded that there is a specific sequence described by the following line:

B1=t{ . . . ,0,0,−1,1,0,0, . . . },

having clear ubiquity: this type of arrhythmia is definitively characteristic and clearly dominant in individuals with HF, with a mean of presence around an order of magnitude greater in HF than in SD or MI, despite the fact that it is also dominant in these subjects. While the primary variabilities (PV) of individuals in the HF and SD databases are large and very similar, what really distinguishes individuals with HF from those recovering from SD is the much higher presence in the former of arrhythmia B1. These arrhythmias correspond to compensated isolated ectopic beats; the present invention does not seek to identify their specific cardiac origin, whether supraventricular or ventricular, since the nature thereof may be associated with another characteristic that has not yet been studied.

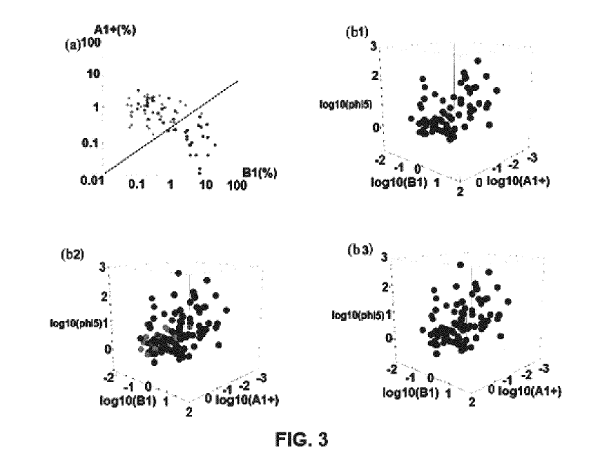

As can be seen, this analysis can provide a new basis for elaborating a general classification based on the inherent and strongly compensated nature of these arrhythmias and their capacity to be reduced to a single universally expressible structure. On the other hand, these are arrhythmias that are relatively rare or absent in individuals in the NSR database. In fact, ectopic beats may occur in the recordings of normal individuals, but they are relatively rare. However, arrhythmias of this type are more present in the recordings of awake individuals at rest and in the supine position (“Fantasia” record mentioned below) than in normal individuals during normal activity. Nevertheless, the prevalence of arrhythmia B1 in HF may require a future review of the diagnostic value (Frolkis et al. 2003; and related references) of the presence of ectopic beats in combination with the capacity to effortlessly adapt to the demands of normal activity (adaptability associated with the presence of arrhythmias A1). Table 1 shows the strong inverse correlation between the presence of compensated ectopic beats (arrhythmias B1) and arrhythmias A1 (±). In addition to Table 1, the opposing presence of arrhythmias A1 and B1 is clearly illustrated in FIG. 3.

Arrhythmia B2 (Regular Paroxysmal Tachycardia):

The graphical representation shows the sequence described by [see FIG. 1 (a, b)]:

B2_{5,1}=t{4,−1,−1,−1}

In many cases an additional line appears [see FIG. 1 (c, d)]:

B2_{5,2}=t{−1,−1,−1,−1}

First of all, the occurrence of these sequences introduces an additional source of randomization upon being combined with A1. This provides an additional adaptive capacity which, on the other hand, may be very limited (it would look like a more intense failure) as the presence of arrhythmias A1 decreases (normal adaptability) in individuals with heart pathologies. Second of all, none of these sequences is compensated. This means that values of N of a higher order must be considered in order to find compensated or finished sequences. In this case, both B2_{5,1} and B2_{5,2} are actually part of the compensated sequence:

B2=t{4,−1,−1,−1,−1}

which actually pertains to a Poincare section of the sixth order (i.e., N=6). It can be seen that compensated sequences are described by lines of the following type:

B2_{N}=t{(N−2),−1, . . . ,−1},

for example:

B2₆=t{4,−1,−1,−1,−1}

B2₇=t{5,−1,−1,−1,−1,−1}

B2₈=t{6,−1,−1,−1,−1,−1,−1}

B2₉=t{7,−1,−1,−1,−1,−1,−1,−1}

etc. All these sequences are almost as present as arrhythmia B1 in HF, although the presence of this type of arrhythmia drops as N increases. This sequence is hardly identifiable if N increases above 10. Actually, the arrhythmia with the greatest presence is B2₆, and that is why this type of arrhythmia is generically referred to as B2 instead of B2₆. The analysis of the presence of this arrhythmia in HF demonstrates that it is as omnipresent as B1, with a different dominance of one over the other, depending on the individual. This sequence is compatible with a regular paroxysmal tachycardia after a pause, which can be attributed to an atrioventricular block, which in some cases gives rise to Stokes-Adams syndrome. Its frequency of presentation in each situation is shown in Table 1. Like arrhythmia B1, arrhythmia B2 is characteristic of HF. It is important to take into account that this arrhythmia represents a noticeable pause, followed by a proportional rapid-rate series to compensate for the pause.

Arrhythmia B3 (Regular Paroxysmal Tachycardia II):

several sequences that are alternatives to B2 can be identified, such as:

B3_{6,1}=t{−1,4,−1,−1,−1}

B3_{6,2}=t{−1,−1,4,−1,−1}

B3_{6,3}=t{−1,−1,−1,4,−1}

B3_{6,4}=t{−1,−1,−1,−1,4},

or:

B3_{7,1}=t{−1,5,−1,−1,−1,−1}

B3_{7,2}=t{−1,−1,5,−1,−1,−1}

B3_{7,3}=t{−1,−1,−1,5,−1,−1}

B3_{7,4}=t{−1,−1,−1,−1,5,−1}

B3_{7,4}=t{1,−1,−1,−1,−1,5}

etc., which can be expressed for a general indicator N as:

B3_{N,i}=t{−1, . . . ,−1,N−2(at position i),−1, . . . ,−1}.

For a given N, all the sequences of B3 with different indicators i have the same presence, but said presence is significantly lower than B2. This sequence could be similar to intermediate situations by joining two consecutive sequences corresponding to regular paroxysmal tachycardia with relative pauses like in arrhythmia B2. It is surprising that this arrhythmia is less characteristic of HF; actually, it is as present in HF as it is in SD, with an almost identical probability distribution. On the other hand, it is significantly more present in NSR than in the individuals with IM and in the individuals in the NSR (“Fantasia”) database.

In some cases, a peculiar sequence appears as “shadows” of arrhythmia B2 [see FIG. 2 (d), for example], which can be identified as lines:

B̅3_{6,1}=t{−4,1,1,1,1}

B̅3_{6,2}=t{1,−4,1,1,1}

B̅3_{6,3}=t{1,1,−4,1,1}

which can generally be written as

B̅3_{N,i}=t{1, . . . ,1,−(N−2)(at position i),1, . . . ,1},

a somewhat complementary sequence of B3_{N-2, i}. A quantitative analysis of this type of arrhythmia is not provided given its complexity.

Arrhythmia A2 (Relating to Breathing):

This is a relatively present sequence, though much less so than arrhythmia B1 or B2 in HF or SD, which appears as the dominant form of sub-arrhythmia in normal or asymptomatic individuals. This compensated sequence can be described by the following line:

A2_{N}=t{sin(2π·j)/N}_{j=1, . . . ,N},

representing a sinusoidal modulation of the heart rate along a range of N beats. This type of arrhythmia with mathematical compensation must also reflect a physiological compensation. Given that it is statistically more frequent with N=5 than with any other order, it may be concluded that it is related to the mean respiratory rate in the human species. Whether or not it is more frequent during sleep than during normal activity will be the object of future studies. Its presence in the records is analyzed in Table 1. It is relatively dominant in NRS with respect to the pathological records, therefore it is significantly less frequent in individuals with HF and SD. It can be deduced that this arrhythmia is, like A1, characteristically non-pathological. In other words, its presence is compatible with a good state of heart health.

The combination of the densities of each identified arrhythmia may constitute a valuable characteristic signature of a specific situation, thereby opening up the pathway to new diagnostic examinations, the meaning of which is out of reach of this example of application of the proposed invention. A fundamental finding is the inverse relation between the presence of certain types of arrhythmia, those which may be considered to be indicative of “health”, and those which may be referred to as “pathological”. Specifically, in the analysis of the public databases used, arrhythmia A1 and arrhythmia B1 are antagonistic: in fact, the relative presence of one with respect to the other is reversed when going from a situation of normality to a pathological state. FIG. 3 shows a universal map in which the presence of both arrhythmias in individuals in the NSR and HF databases is analyzed. A very clear difference can be seen between NSR and HF, based on the presence of A1 and B1.

Furthermore, the value of the primary variability Φ_{N} completes the set of characteristic variables for providing the distinctive, characteristic seal of each situation: it must be noted in Table 1 that the combinations of the presences of each arrhythmia and the PV makes up a unique and highly differentiated signature of each situation. The difference between MI and SD is Φ_{N}, small for MI and large for SD. The multivariable probability density function for each situation in the space of variables {log₁₀(A1),log₁₀(B1),log₁₀(Φ_{N})} would be determined by the density of the points where, in the space, it would correspond to each situation, as can be seen in FIG. 3.

The clinical value of this representation could be extremely significant. In fact, the therapeutic effect of drugs with a specific cardiovascular action, targeting objectives such as sympathetic/parasympathetic axis (adrenergic beta-blockers), perhaps the neurohormonal rennin-angiotensin-aldosterone axis (IECAs, ARA II, etc.), etc., could give rise to the modification of the relative presence of each arrhythmia and displace the location of the corresponding graph in the direction of the NSR region. The potential prognostic value of the results obtained when applying the method of the present invention to HR is obvious. The observation of the four-dimensional graphical representation of HR allows the easy and immediate identification of disturbances in normality in healthy people.

Future clinical studies will expand the depth of knowledge acquired with the analysis proposed in this invention, such as the identification of new characteristics and new general arrhythmic patterns, relating to other pathologies and situations, not necessarily of a cardiac origin, for example, diabetes, hypertension, hypothyroidism, or even psychic disturbances.

TABLE 1

| Ar- | NSR(normal | | | | |
|---|---|---|---|---|---|
| rhythmia | activity) | “Fantasia” | MI | SD | HF |
| A1+ | 1.21849 | 0.841977 | 0.682145 | 0.355535 | 0.23905 |
| A1− | 0.725413 | 0.657101 | 0.502548 | 0.264764 | 0.188382 |
| A2+ | 0.417186 | 0.576313 | 0.320071 | 0.33573 | 0.158211 |
| A2− | 0.630867 | 0.58272 | 0.379129 | 0.284021 | 0.175146 |
| B1 | 0.354003 | 0.568972 | 1.7985 | 1.48564 | 5.59225 |
| B2 | 0.431622 | 0.32933 | 0.339216 | 0.493797 | 0.772125 |
| Φ₅ | 10.1739 | 2.4295 | 1.7567 | 41.2053 | 48.9619 |

In conclusion, the present invention proposes a quantitative method not only to provide a universal representation of heart rate variability, but also a clinical tool that is potentially useful in evaluating heart function, risks, and probably other related health issues. In the present invention, among the many different universal sequence types that probably exist, some patterns that stand out have been described mathematically with a relatively simple structure, identifying five general types of arrhythmias. The databases used allowed identifying the characteristic signatures of individuals included in the NSR, MI, HF, and SD databases, by relating these basic types of arrhythmias with the different situations of heart function. As a fundamental result, it has been quantitatively demonstrated that two of these arrhythmias are characteristic of the state of health, whereas the other three are pathological. Their relative presence in an individual may eventually be related to specific situations, with the growing clinical evidence provided by this methodology which will be built on in the future. Furthermore, the temporal evolution of arrhythmic structures in a patient, which are visible with the application of the methodology of the invention, can provide very valuable information about the state and/or clinical evolution of said patient. It is considered a methodology that is easy to apply, and the potential of its results in non-invasive clinical diagnosis and prognosis could be vital.

1. A method for obtaining data associated with a heart rate variability (HRV) pattern, comprising:
a) measuring and recording a number M of consecutive time intervals {Xᵢ}_{i=1, . . . ,M} corresponding to cycles of one or more components of a heart “pQRSt” complex of an electrocardiogram, with a precision equal to or greater than 10% of the mean value of the cycle time, and M being greater than 2;
b) calculating the variability on said M intervals of a sequence of consecutive vectors {δ}_{j=1, . . . ,M-N} of N components, according to the algorithm or transformation defined by the expression:
$${δ}_{j} = {\{ {\sum }_{n = 0}^{m} ( \begin{matrix}m \\ n\end{matrix} ) {( - 1 )}^{n} ( \begin{matrix}{〈 X 〉}_{{N}_{0} , j \cdot {ς}_{0} + {K}_{0}}^{- 1} {X}_{j + n + k \cdot {ɛ}_{0} + {J}_{0}} - \\ {ɛ}_{1} {〈 X 〉}_{{N}_{1} , j \cdot {ς}_{1} + {K}_{1}}^{- 1} {〈 X 〉}_{{N}_{2} , j + n + k \cdot {ɛ}_{2} + {K}_{2}}\end{matrix} ) \}}_{k = {J}_{1} , … , {J}_{1} + N - 1} ,$$
and the following notation:
$${〈 X 〉}_{L , l} = {L}^{- 1} {\sum }_{h = 0}^{L - 1} {X}_{l + h} , with ( \begin{matrix}m \\ n\end{matrix} ) = \frac{m !}{n ! ( m - n ) !} ,$$
where the following parameters are integers and their selection determines the final form of the mentioned transformation:
{m,N,N₀,N₁,N₂,ε₀,ε₁,ε₂,ζ₀,ζ₁,J₀,J₁,K₀,K₁,K₂},
where additionally:
m is a natural indicator representing the order of the discrete variation that is calculated;
N is the dimension or number of components of each vector δⱼ, where N≥2;
N₀, N₁, and N₂ indicate the number of values that are used for calculating the corresponding indicated local average in the general formula of the algorithm;
ε₀, ε₁, ε₂ have binary values 0 or 1, and indicate if the corresponding elements are, respectively, fixed or mobile in the calculation of each of the components of the vector δⱼ;
ζ₀, ζ₁ have binary values 0 or 1, and indicate if the local mean is, respectively, fixed or mobile;
J₀ and J₁ indicate the delay or advance of the first element that is taken in the calculation on the basis of the indicator j;
K₀, K₁, K₂ indicate the delay or advance of the first element that is taken in the corresponding local series for calculating the indicated local average;
and wherein the position of the point indicated by the values of the components of each of the vectors δⱼ is graphically represented in two or more dimensions.

2. The method according to claim 1, comprising an additional step of comparing the data obtained with behavioral patterns associated with a vector function A={aⱼ}_{j=1, . . . ,N}, corresponding to the parameterization of a heart sequence, where the elements aⱼ are fixed values or functions of one or more variables, and where an additional step of comparing the useful data obtained with said function A is performed according to the following steps:
a) calculating the general angle θᵢ, the cosine of which is determined by:
$$cos ( {θ}_{i} ) = \frac{A \cdot {δ}_{i}}{‖ A ‖ \cdot ‖ {δ}_{i} ‖} ,$$
where the symbol ∥ ∥ is the general norm of a vector in N dimensions, such that:
$$‖ A ‖ = {( {\sum }_{j = 1}^{N} {a}_{j}^{2} )}^{1 / 2} ;$$
b) calculating the number of events M′ such that the angle θᵢ is less than a predetermined tolerance ε, where 0<ε<1, such that the function A is explored in its space of existence in order to find said events in which θᵢ<ε;
c) calculating the coefficient M′/M.

3. The method according to claim 2,
wherein the calculation of the series {δⱼ}_{j=1, . . . ,M-N} of consecutive vectors of N dimensions or components is performed according to the following definitions of parameters:
$$\{ \begin{matrix}m = 0 \\ N = 5 \\ {N}_{0} = 5 \\ {N}_{1} = 5 \\ {N}_{2} = 5 \\ {ɛ}_{0} = 1 \\ {ɛ}_{1} = 1 \\ {ɛ}_{2} = 0 \\ {ς}_{0} = 1 \\ {ς}_{1} = 1 \\ {J}_{0} = 0 \\ {J}_{1} = 0 \\ {K}_{0} = 0 \\ {K}_{1} = 0 \\ {K}_{2} = 0\end{matrix} \} ,$$
such that the definition of the variability on said M intervals is:
δⱼ={Xⱼ₊ₖ [symbol] X [symbol] _{N,j}⁻¹−1}_{k=0, . . . ,N-1}.

4. The method according to claim 3, wherein the following steps are additionally performed:
a) calculating the number of events on the basis of the series {Xᵢ}_{i=1, . . . ,M} with the specific definition of the vector A=t{(N+1)/2−j}_{j=1, . . . ,N} where N>1;
b) using the indicator m_{S1}/M, with m_{S1}=M′ calculated in the preceding point, for determining the existence of behavioral patterns associated with the vector function A.

5. The method according to claim 3, wherein the following steps are additionally performed:
a) calculating the number of events M′ on the basis of the series {Xᵢ}_{i=1, . . . ,M} with the specific definition of the vector A_{N}=t{sin(2π·j)/N}_{j=1, . . . ,N} , where N can range from N=3 to N=12, corresponding to a sinusoidal modulation of the heart rhythm combined with the respiratory rhythm, where t can have any value;
b) using the indicator m_{S2}/M, with m_{S2}=M′ calculated in the preceding point, for determining the existence of behavioral patterns associated with the vector function A.

6. The method according to claim 3, wherein the following steps are additionally performed:
a) calculating the coefficient M′/M on the basis of the series {Xᵢ}_{i=1, . . . ,M} with the definition of the vector
$${A}_{N} = t \{ - 1 , 1 , \underbrace{0 , … , 0}_{N} \} ,$$
where N can range from N=1 to N=20, corresponding to a compensated ectopic beat, and where t can have any value;
b) using the indicator m_{E}/M, with m_{E}=M′ calculated in the preceding step, for determining the existence of behavioral patterns associated with the vector function A_{N}.

7. The method according to claim 3, wherein the following steps are additionally performed:
a) calculating the coefficient M′/M on the basis of the series {Xᵢ}_{i=1, . . . ,M} with the specific definition of the vector
$${A}_{N} = t \{ N , \underbrace{- 1 , … , - 1}_{N} \} ,$$
where N can range from N=2 to N=20, corresponding to a regular paroxysmal tachycardia, and where t can have any value;
b) using the indicator m_{TP}/M, with m_{TP}=M′ calculated in the preceding point, for determining the existence of behavioral patterns associated with the vector function A_{N}.

8. The method according to claim 1, wherein the component of the heart “pQRSt” complex is the RR interval of an electrocardiogram.

9. The method according to claim 1, wherein the recording of a number M of consecutive time intervals {Xᵢ}_{i=1, . . . ,M}, corresponding to cycles of a component of a heart “pQRSt” complex is performed with a precision greater than 0.01% of the mean value of the cycle time.

10. The method according to claim 3, wherein 0<ε<0.1.

11. The method according to claim 6, wherein t is 1 or −1.
